(19) United States
(12) Patent Application Publication
(10) Pub. No.: US 2012/0316605 A1
(43) Pub. Date: Dec. 13, 2012

(54) Taper Lock For A Polyaxial Spinal Rod Screw Assembly

(76) Inventor: Greg Palagi, Tinley Park, IL (US)

(21) Appl. No.: 13/495,463

(22) Filed: Jun. 13, 2012

Related U.S. Application Data
(60) Provisional application No. 61/496,112, filed on Jun. 13, 2011.

(51) Int. Cl. A61B 17/86

(52) U.S. Cl. 606/305

(57) ABSTRACT

A collet for a spinal rod bone screw assembly is configured to provide an interference fit with the top or head of a polyaxial bone screw in order to aid in locking orientation of a polyaxial bone screw head relative to the polyaxial bone screw. An increase in spherical coverage created by the configuration of the collet increases the amount of surface contact with the polyaxial bone screw head. This allows for more controlled, uniform and secure orientation of the polyaxial head with respect to the bone screw during lock-up. A resilient, tapered base of the collet has a plurality of slots that allow the end of the collet to splay outwardly over and upon the head of the bone screw to create a snap or frictional interference fit between the collet and the polyaxial bone screw head when a spinal rod is fixed in the polyaxial bone screw head.

### RELATED APPLICATIONS

This patent application claims the benefit of and/or priority under 35 U.S.C. §119(e) to U.S. Provisional Patent Application Ser. No. 61/496,112 filed Jun. 13, 2011, entitled “Taper Lock For A Polyaxial Spinal Rod Screw Assembly” the entire contents of each of which is specifically incorporated herein by this reference.

### BACKGROUND OF THE INVENTION

1. Field of the Invention

The present invention relates to screw assemblies for spine fixation components, constructs and assemblies and, more particularly, to components for screw assemblies for spinal rod applications.

2. Background Information

Spinal orthopedic assemblies and constructs such as spine plates, spinal bone screw assemblies for spinal rods and other devices (spinal components) have made a profound contribution to the correction of spinal deformities, accidents and other problems in the cervical as well as thoracic, lumbar and sacral spine. These and other spinal devices are typically fixed to vertebrae using vertebral bone screws. Vertebral bone screws are specially designed and manufactured bone screws that are placed into the bone of a vertebra. Vertebral bone screws placed in the vertebra offer superior strength and pull-out resistance as compared to other forms of fixation in spine surgery. The ability to achieve vertebral fixation has allowed surgeons to obtain more secure fixation of the spinal components involved, which permits more powerful correction of spine problems and reported better clinical outcomes.

In addition to other uses, bone screws provide a solid foundation for the attachment of spinal rods. Spinal rods are used for the fixation of a plurality of vertebrae for various situations. A spinal rod is held relative to the vertebrae by a spinal rod screw assembly. Various types of spinal rod screw assemblies are known such as those that allow for inter-operative adjustments in the coronal, transverse and sagittal planes. Certain spinal rod screw assemblies allow for various degrees of freedom of attachment of a spinal rod thereto from any direction, angle, and height. In all cases, however, the spinal rod screw assemblies hold a spinal rod and are fixed to a vertebra. Spinal rods can thus be rigidly locked into a variety of positions along with other types of implant components. This allows a surgeon to tailor-make each construct for the individual case.

One type of spinal rod screw assembly is known as a polyaxial tulip head spinal rod holder. The polyaxial tulip head spinal rod holder includes a polyaxial bone screw, a polyaxial tulip head and a taper lock. The polyaxial tulip head is situated about the polyaxial bone screw head while the taper lock is situated within the polyaxial tulip head. Downward pressure exerted against the taper lock during installation causes the taper lock to bind against the polyaxial bone screw head and between polyaxial tulip head such that the orientation of the tulip head is fixed relative to the bone screw. While current taper locks are functional, there is room for improvement.

Thus, there is a need for an improved taper lock for a spinal rod screw assembly.

### SUMMARY OF THE INVENTION

The present invention is a polyaxial bone screw assembly and/or collet of a polyaxial bone screw assembly, for holding a spinal rod in a fixed spatial orientation relative to the spine, the collet configured for reception in a polyaxial bone screw head of the polyaxial bone screw assembly, provide an interference or snap-on fit connection with a head of a polyaxial bone screw of the polyaxial bone screw assembly, and fix orientation of the polyaxial bone screw head relative to the polyaxial bone screw when a spinal rod is fixed to the polyaxial bone screw head. The collet has a tapered end or base that wedges between the head of the polyaxial bone screw and an inside surface of an interior of the polyaxial bone screw head during spinal rod lock-up (fixation). An increase in spherical coverage created by the present configuration increases the amount of surface contact with the polyaxial bone screw head. This allows for more controlled, uniform and secure fixation of the orientation of the polyaxial head holding the spinal rod with respect to the bone screw.

The tapered end of the collet or “taper lock” has a plurality of cuts, slots, cutouts or the like create a resiliency to the base which allows the polyaxial bone screw head to snap into the base thus providing a frictional interference fit. The plurality of cutouts in the base allows the resilient base to splay slightly during reception of the polyaxial bone screw head, then conform about the polyaxial bone screw head.

In one embodiment, the peripheral circumference of the base of the present taper lock has a cutout spaced every 60 degrees. Other spacing, of course, may be used such as pairs of cutouts oriented about the end relative to themselves and others. Various shapes of cutouts may also be used other than that shown.

### BRIEF DESCRIPTION OF THE DRAWINGS

The above mentioned and other features, advantages and objects of this invention, and the manner of attaining them, will become apparent and the invention itself will be better understood by reference to the following description of an embodiment of the invention taken in conjunction with the accompanying drawings, wherein:

FIG. 1 is an exploded view of components of a polyaxial bone screw assembly for holding a spine rod in a fixed relationship with the spine, the polyaxial bone screw assembly utilizing a collet or taper lock fashioned in accordance with the present principles;

FIG. 2 is a view of the present collet received onto the polyaxial bone screw of the polyaxial bone screw assembly of FIG. 1;

FIG. 3 is a side view of the present collet;

FIG. 4 is an upper side view of the present collet;

FIG. 5 is a sectional side view of the present collet situated on the head of the polyaxial bone screw of the polyaxial bone screw assembly of FIG. 1; and

FIG. 6 is a sectional side view of the present collet situated on the head of the polyaxial bone screw within the interior of the polyaxial bone screw head of the polyaxial bone screw assembly of FIG. 1.

Like reference numerals indicate the same or similar parts throughout the several figures.

A description of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF THE INVENTION

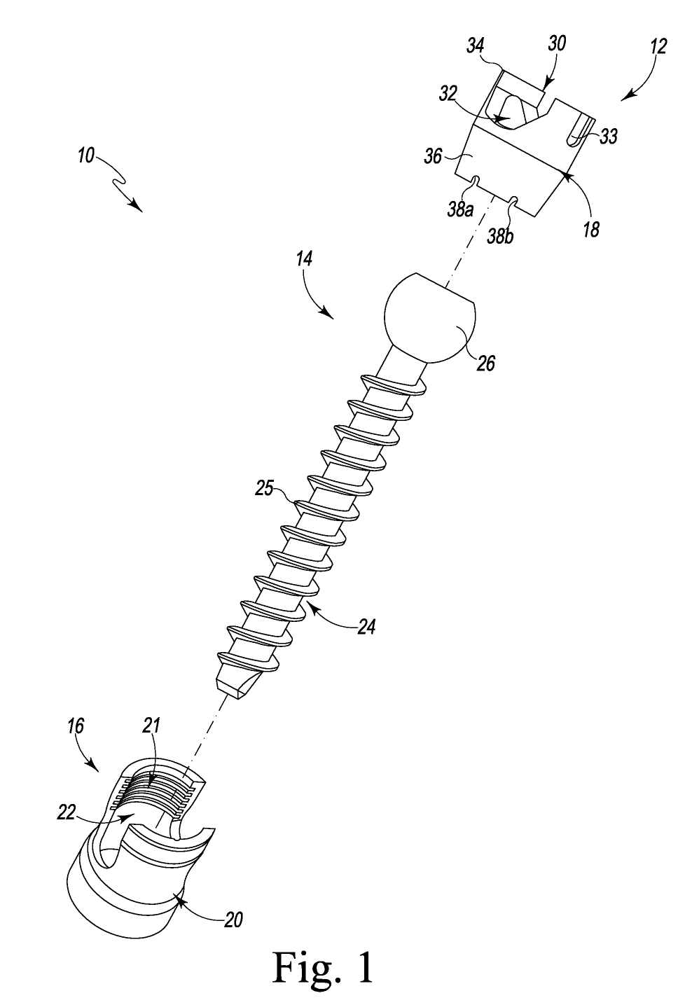

Referring to FIG. 1, there is depicted a polyaxial bone screw assembly, generally designated 10, utilizing a collet or taper lock 12 fashioned in accordance with the present principles, the polyaxial bone screw assembly 10 for holding a spinal rod (not shown) relative to and on a spinal bone (e.g. a vertebra—not shown). FIG. 1 depicts primary components of the polyaxial bone screw assembly 10 in an exploded or pre-assembled view. The polyaxial bone screw assembly 10 is characterized by polyaxial spinal rod holder, connector or head 16, a polyaxial bone screw 14, and a collet or taper lock 12.

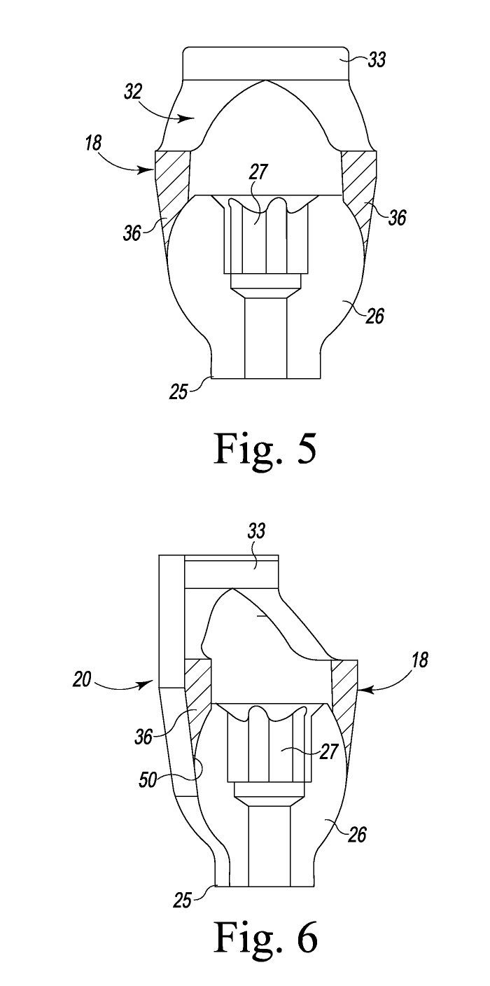

The polyaxial bone screw 14 is characterized by body 24 having a threaded shank or shaft 25 and a polyaxial head 26. As seen in FIGS. 5 and 6, the polyaxial head 26 has a bore 27 that is configured to receive a bone screw installation tool (not shown). In one form, the bore 27 is hexalobe configured to receive a hexalobe bone screw installation tool.

The polyaxial head 16 is defined by a generally tulip shaped body 20, it being understood that the body 20 may take other shapes as appropriate. The body 20 has an interior bore 21 extending from a top of the body 20 to a bottom of the body 20. The bore 21 receives the bone screw 14 and the collet 12. A spinal rod reception area 22 is defined in the body 20, formed as a first pocket, cutout or notch between first and second sides of the body and a second pocket, cutout or notch between the first and second sides of the body 20. The first and second notches are formed to receive a spinal rod therein and thus are generally arcuate or cup-shaped. The first and second sides also include internal threading for reception of a set screw or the like (not shown) that is used to “lock up” the polyaxial head 16 on the bone screw 14 via the collet 12 (i.e. during fixation or lock-up of a spinal rod (not shown) into the taper lock/polyaxial head assembly).

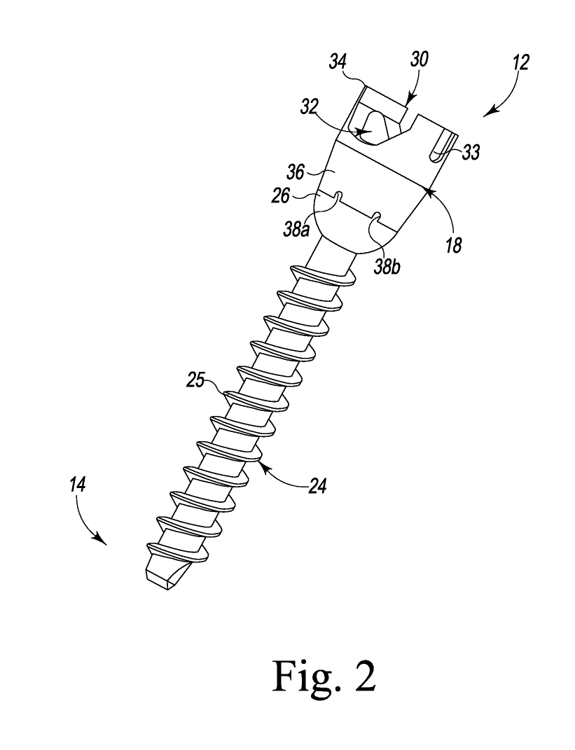

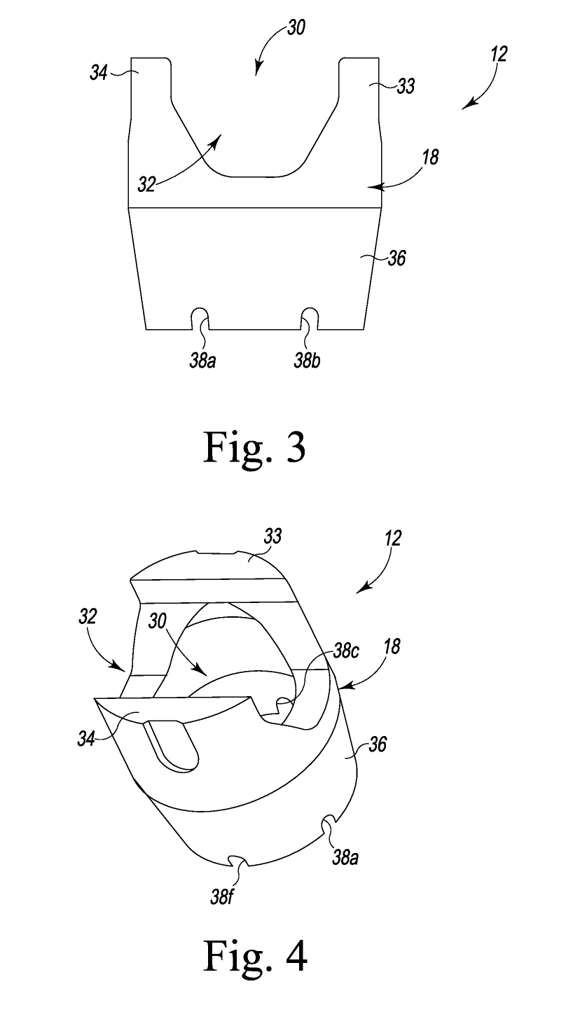

The present collet or taper lock 12 is shown in FIG. 2 received on the polyaxial bone screw 14 but without the polyaxial head 16. Referring to FIGS. 3 and 4, the collet 12 is shown. The collet 12 is defined by a body 18 having a base or skirt 36 on a lower portion thereof and first and second upstanding flanges 33, 34 that define a first pocket, cutout or notch between first and second flanges 33, 34 and a second pocket, cutout or notch between the first and second flanges 33, 34. The first and second notches are formed to receive a spinal rod therein and thus are generally arcuate or cup-shaped. An interior bore 30 extends from a top of the body 18 to a bottom of the body 18. The bore 30 allows access to the polyaxial head 26 of the polyaxial bone screw 14.

In accordance with the principles of the present invention, the bottom of the base 36 includes a plurality of cuts, cutouts, notches or the like 38a-38f. Each cutout 38a-38f is preferably, but not necessarily, spaced 60° about the bottom of the base 36. Other numbers of cutouts and orientations may be used. The cutouts 38a-38f create a collet mechanism that allows the polyaxial screw head 26 to snap into the base 36 of the collet 12 thus creating a frictional interference fit. It should be appreciated that the number of cutouts 38 in the base 36 may vary from two (2) to as many as desired. In the preferred embodiment though, there are six (6) cutouts. It is also preferable, but not necessary, that the cutouts 38 are evenly spaced about the bottom of the base 36. The cutouts 38 create an interference or snap on fit with the polyaxial screw head 26 by allowing the bottom of the base 36 to splay slightly then from back around the screw head 26 since the base 36 is resilient. The spherical coverage of the head 26 increases the amount of surface contact between the base 36 of the collet 12 and the polyaxial screw head 26.

FIG. 5 shows the collet 12 situated on the polyaxial screw head 26 in order to illustrate the increase in contact between the base 36 of the collet 12 and the polyaxial screw head 26 relative to the prior art. Whereas prior art collets/taper locks generally cover or contact only the upper portion of the screw head 26, the present collet extends about and over at least half of the polyaxial screw head 26. This allows for more controlled, uniform, and secure orientation of the polyaxial screw head 16 with respect to the polyaxial bone screw 14.

FIG. 6 illustrates the manner in which the collet 12 achieves lock-up of it and the polyaxial head 20 relative to the head 26 of the polyaxial bone screw 24, particularly during lock-up of the spine/spinal rod (not shown) into the polyaxial bone screw head 20. As a set screw (not shown) or the like, is received in the polyaxial bone screw head 20 (after receipt of the spinal rod into the polyaxial bone screw head 20 and collet 18) the spinal rod forces the collet 12 onto the head 26 of the polyaxial bone screw 24 which splays the end or base 36 thereof. The splayed end 36 abuts the interior 50 of the polyaxial bone screw head 20 and wedges against and between the head 26 of the bone screw 24 and the polyaxial head 20 to lock up or fix the orientation of the polyaxial bone screw head 20 relative to and on the head 26 of the polyaxial bone screw 24. This creates an interference or wedge fit. The coupling of the collet 12 to the head 26 of the polyaxial bone screw 24 is accomplished via an interference or snap fit.

The various components of the polyaxial bone screw assembly 10 and thus the present collet/taper lock 12 are made from a bio-compatible material such as stainless steel or titanium. Other bio-compatible materials, or course, may be used.

While the invention has been illustrated and described in detail in the drawings and foregoing description, the same is to be considered as illustrative and not restrictive in character, it being understood that only a preferred embodiment has been shown and described and that all changes and modifications that come within the spirit of the invention are desired to be protected.

What is claimed is:

1. A polyaxial bone screw assembly for holding a spinal rod in a fixed spatial orientation relative to a spinal bone, the polyaxial bone screw assembly comprising:
a polyaxial bone screw having a threaded shank and a rounded head;
a polyaxial bone screw head disposed on the rounded head of the polyaxial bone screw and configured to swivel about the rounded head of the polyaxial bone screw before the polyaxial bone screw head is fixed in spatial orientation relative to the polyaxial bone screw, and to receive and hold a spinal rod once the spatial orientation of the polyaxial bone screw head is fixed, the polyaxial bone screw head having a bore extending from one end of the polyaxial bone screw head to another end of the polyaxial bone screw head and defining an interior within the polyaxial bone screw head; and
a collet disposed in the interior of the polyaxial bone screw head, the collet having a first end resting upon the rounded head of the polyaxial bone screw and configured to splay about the rounded head of the polyaxial bone screw and fix the spatial orientation of the polyaxial bone screw head through interference between the first end, the rounded head of the polyaxial bone screw, and the polyaxial bone screw head when a spinal rod is received in and affixed to the polyaxial bone screw head.

2. The polyaxial bone screw assembly of claim 1, wherein the first end of the collect includes a plurality of cutouts.

3. The polyaxial bone screw assembly of claim 2, wherein each one of the plurality of cutouts extend axially from the first end of the collet towards a second end of the collet.

4. The polyaxial bone screw assembly of claim 2, wherein the plurality of cutouts is situated about the first end in diametrically opposite pairs.

5. The polyaxial bone screw assembly of claim 2, wherein the plurality of cutouts comprises six.

6. The polyaxial bone screw assembly of claim 1, wherein the first end of the collect is tapered inwardly.

7. The polyaxial bone screw assembly of claim 6, wherein the first end of the collect includes a plurality of cutouts.

8. The polyaxial bone screw assembly of claim 7, wherein each one of the plurality of cutouts extend axially from the first end of the collet towards a second end of the collet.

9. The polyaxial bone screw assembly of claim 7, wherein the plurality of cutouts is situated about the first end in diametrically opposite pairs.

10. The polyaxial bone screw assembly of claim 9, wherein the plurality of cutouts comprises six.

11. A polyaxial bone screw assembly for holding a spinal rod in a fixed spatial orientation relative to a spinal bone, the polyaxial bone screw assembly comprising:
a polyaxial bone screw having a threaded shank and a rounded head;
a polyaxial bone screw head disposed on the rounded head of the polyaxial bone screw and configured to swivel about the rounded head of the polyaxial bone screw before the polyaxial bone screw head is fixed in spatial orientation relative to the polyaxial bone screw, and to receive and hold a spinal rod in spinal rod grooves disposed in a first end of the polyaxial bone screw head once the spatial orientation of the polyaxial bone screw head is fixed, the polyaxial bone screw head having a bore extending from the first end to a second end of the polyaxial bone screw head that also defines an interior within the polyaxial bone screw head; and
a collet disposed in the interior of the polyaxial bone screw head, the collet having a tapered first end resting upon the rounded head of the polyaxial bone screw and having a plurality of cutouts configured to allow the tapered first end of the collet to splay about the rounded head of the polyaxial bone screw and fix the spatial orientation of the polyaxial bone screw head through interference between the tapered first end, the rounded head of the polyaxial bone screw, and the polyaxial bone screw head when a spinal rod is received in and affixed to the polyaxial bone screw head.

12. The polyaxial bone screw assembly of claim 11, wherein each one of the plurality of cutouts extend axially from the tapered first end of the collet towards a second end of the collet.

13. The polyaxial bone screw assembly of claim 12, wherein the plurality of cutouts is situated about the tapered first end in diametrically opposite pairs.

14. The polyaxial bone screw assembly of claim 12, wherein the plurality of cutouts comprises six.

15. A polyaxial bone screw assembly for holding a spinal rod in a fixed spatial orientation relative to a spinal bone, the polyaxial bone screw assembly comprising:
a polyaxial bone screw having a threaded shank and a rounded head;
a polyaxial bone screw head disposed on the rounded head of the polyaxial bone screw and configured to swivel about the rounded head of the polyaxial bone screw before the polyaxial bone screw head is fixed in spatial orientation relative to the polyaxial bone screw, and to receive and hold a spinal rod in spinal rod grooves disposed in a first end of the polyaxial bone screw head once the spatial orientation of the polyaxial bone screw head is fixed, the polyaxial bone screw head having a bore extending from the first end to a second end of the polyaxial bone screw head that also defines an interior within the polyaxial bone screw head; and
a collet disposed in the interior of the polyaxial bone screw head, the collet having a tapered base resting upon the rounded head of the polyaxial bone screw and having a plurality of cutouts configured to allow the tapered base of the collet to splay about the rounded head of the polyaxial bone screw and fix the spatial orientation of the polyaxial bone screw head through interference between the tapered base, the rounded head of the polyaxial bone screw, and the polyaxial bone screw head when a spinal rod is received in and affixed to the polyaxial bone screw head.

16. The polyaxial bone screw assembly of claim 15, wherein each one of the plurality of cutouts extend axially from the tapered base of the collet towards a second end of the collet.

17. The polyaxial bone screw assembly of claim 15, wherein the plurality of cutouts is situated about the tapered base in diametrically opposite pairs.

18. The polyaxial bone screw assembly of claim 15, wherein the plurality of cutouts comprises six.
